(19) United States
(12) Patent Application Publication
(10) Pub. No.: US 2022/0298319 A1
(43) Pub. Date: Sep. 22, 2022

(54) Insoluble Polysaccharide Foams

(71) Applicant: The Penn State Research Foundation, University Park, PA (US)

(72) Inventors: Jeffrey M. Catchmark, State College, PA (US); Ke Liu, University Park, PA (US); Kai Chi, Cedar Grove, NJ (US); Jingxuan Yang, University Park, PA (US)

(21) Appl. No.: 17/631,255

(22) PCT Filed: Jul. 29, 2020
(86) PCT No.: PCT/US20/44061

§ 371 Date: Jan. 28, 2022

Related U.S. Application Data
(60) Provisional application No. 62/879,849, filed on Jul. 29, 2019.

(51) Int. Cl. C08J 9/00; C08J 9/228; C09D 103/02

(52) U.S. Cl. CPC C08J 9/0061; C08J 9/228; C09D 103/02; C08J 2303/02; C08J 2405/08

(57) ABSTRACT

An insoluble foam composite material is formed by a mixture combining an anionic polysaccharide, a cationic polysaccharide, a solvent, and a plasticizer. In particular, the composite material can be prepared by heating, freezing and lyophilizing the mixture to produce, for example, insoluble porous foam-like composites.

### STATEMENT AS TO FEDERALLY SPONSORED RESEARCH

This invention was made with government support under Grant No. W81XWH-18-1-0150 awarded by the U.S. Army, under Grant No. DM160335 awarded by the U.S. Army and under Hatch Act Project No. PEN04602 awarded by the United States Department of Agriculture. The Government has certain rights in the invention.

### TECHNICAL FIELD

The present disclosure relates to an insoluble foam composite material that can be formed by a mixture combining an anionic polysaccharide, a cationic polysaccharide, a solvent, and a plasticizer. In particular, the composite material can be prepared by heating, freezing and lyophilizing the mixture.

### BACKGROUND

Insoluble low density, porous materials, such foams, are needed for a wide variety of commercial applications including insulation materials, packaging materials, absorbent materials for applications ranging from personal hygiene to liquid hazardous waste remediation or removal, porous materials for biomedical applications including wound care and tissue regeneration, and, if edible, materials for food production such as ‘puffed’ food products or diet food products. Furthermore, materials which are compostable offer improved sustainability as they can be disposed safely in landfills or even used as an energy source through processes such as anaerobic digestion.

Starch, a natural biopolymer found in plants such as corn or potato, has been extensively utilized to develop expandable or so-called ‘puffed’ materials with other ingredients. Glenn et al. (U.S. Pat. No. 5,958,589, 1995) developed a starch-based microcellular foam using a novel solvent exchange method, and they claimed that such a material had superior properties such as improved mechanical strength, high pore volume, and low density. A starch-lignin foam was prepared by Stevens et al. (2010), which showed that a 20% replacement of starch with lignin had no adverse effect on foam density and morphology. More recently, Dougherty et al. (U.S. patent application #2010/0189843, 2010) invented a hydroxypropylated starch to improve the extrusion process of a food composite whereby the hydroxypropylated starch aids in the retention of dietary fiber contained in the composite. Also, other biopolymers such as carboxymethyl cellulose and xanthan gum have been applied to expand with starch, which is said to improve the shape, texture and structure of starch-based composite (Gimeno, Moraru, and Kokini, 2004). Starch composites which consist of principally biologically derived polymers, however, are typically soluble in polar solutions, limiting their use in many applications. Thus, a need exists to create insoluble composites such as insoluble starch composite with high liquid absorbing capability and desirable mechanical properties.

### SUMMARY

An advantage of the present invention is a composite material that can be used for a variety of applications. Advantageously, the composite is insoluble in liquid environments but also imparts desirable mechanical strength and liquid barrier properties.

These and other advantages are satisfied, at least in part, by a composite material such as a foam-like porous material composition which is insoluble in liquid environments. Advantageously the composite material, depending on the composite processing and the plasticizer type present in the composition, is capable of staying intact when immersed in water. In some cases, the composition is completely insoluble under a desired pH condition (e.g., from a pH of about 2 to about 13).

In one implementation, an insoluble composite material is formed by:

creating a mixture of at least one anionic polysaccharide, at least one cationic polysaccharide, at least one plasticizer, and at least one solvent;

heating the mixture;

pouring the mixture into a mold;

freezing the mixture; and

lyophilizing the mixture.

In a second implementation, an insoluble composite material is formed by:

creating a mixture containing at least one anionic polymer, at least one cationic polymer, and at least one solvent;

exposing the mixture to an elevated temperature to reduce the content of the solvent to solidify the mixture;

soaking the solid mixture in a second solution containing a plasticizer;

freezing the mixture; and

lyophilizing the mixture.

In a third implementation, an insoluble composite material is formed by:

creating a mixture containing at least one anionic polysaccharide, at least one cationic polysaccharide, and at least one solvent;

exposing the mixture to an elevated temperature to reduce the content of the solvent to solidify the mixture;

soaking the solid mixture in a second solution containing a plasticizer;

freezing the mixture; and

lyophilizing the mixture.

In a fourth implementation, an insoluble composite material is formed by:

creating a mixture containing at least one non-gelatinized anionic starch, at least one chitosan, and at least one aqueous solvent;

exposing the mixture to an elevated temperature to gelantinize the starch and reduce the content of the solvent to solidify the mixture;

soaking the solid mixture in a second solution containing a plasticizer;

freezing the mixture; and

lyophilizing the mixture.

In a fifth implementation, an insoluble composite material is formed by:

creating a mixture containing at least one non-gelatinized anionic starch, at least one chitosan, and at least one aqueous solvent;

heating the mixture to partially gelantinize the at least one non-gelatinized anionic starch;

pouring the mixture into a mold;

freezing the mixture; and

lyophilizing the mixture.

In a sixth implementation an insoluble composite material is formed by:

creating a mixture containing at least one anionic polysaccharide, at least one cationic polysaccharide, and at least one solvent;

heating the mixture to reduce solvent content in the mixture and to form a solid mixture;

soaking the solid mixture in a second solution containing a plasticizer and a charged polysaccharide;

freezing the mixture; and

lyophilizing the mixture.

In a seventh implementation, an insoluble composite material is formed by:

creating a mixture containing at least one anionic polysaccharide, at least one cationic polysaccharide, and at least one solvent;

heating the mixture to reduce solvent content in the mixture and to form a solid mixture;

soaking the solid mixture in a second solution containing a plasticizer and an antiseptic agent (e.g., antiseptic molecule such as PHMB);

freezing the mixture; and

lyophilizing the mixture.

In an eighth implementation, an insoluble composite or coating material includes a mixture of at least one anionic polysaccharide, at least one cationic polysaccharide, and a solvent and a plasticizer.

In some embodiments, the plasticizer can be a molecule that contains carbon, oxygen and hydrogen; a molar mass between 60 and 95, have a boiling point between 150° C. and 300° C., have at least one —OH group; at least one CH2 group, and/or is nontoxic to humans.

In some embodiments, the plasticizer can be a molecule that contains carbon, oxygen and hydrogen; a molar mass between 60 and 95: a boiling point between 150° C. and 300° C.; at least one —OH group; at least one CH3 group; and/or is nontoxic to humans. In some embodiments, the plasticizer can be glycerol, propylene glycol or combinations thereof.

In a ninth implementation, a method for producing a composite or coating composition includes:

combining one or more anionic polysaccharides, one or more cationic polysaccharides, a plasticizer, and a solvent to obtain a solution,

heating the solution,

freezing the mixture; and

lyophilizing the mixture.

In some embodiments, the one or more anionic polysaccharides can include at least one anionic starch. In some embodiments, said at least one anionic starch can include amylopectin. In some embodiments, the anionic starch can be selected from the group consisting of amylopectin, amylose, and combinations thereof. In some embodiments, the anionic starch can include at least 70% w/w amylopectin and at least 20% w/w amylose. In some embodiments, said one or more cationic polysaccharides can be chitosan. The ratio of said one or more anionic polysaccharides to said one or more cationic polysaccharides can be between about 10:1 to about 50:1. The ratio of said one or more anionic polysaccharides to said one or more cationic polysaccharides can be at least 20:1. In some embodiments, said plasticizer comprises glycerol, propylene glycol or combinations thereof.

Additional advantages of the present invention will become readily apparent to those skilled in this art from the following detailed description, wherein only the preferred embodiment of the invention is shown and described, simply by way of illustration of the best mode contemplated of carrying out the invention. As will be realized, the invention is capable of other and different embodiments, and its several details are capable of modifications in various obvious respects, all without departing from the invention. Accordingly, the drawings and description are to be regarded as illustrative in nature, and not as restrictive.

### DESCRIPTION OF DRAWINGS

FIG. 1 provides a compressive stress-strain curves of plasticized starch foams formed using Method 1 described herein.

FIGS. 2A-2B provide compressive stress-strain curves of plasticized starch foams formed using Method 2 described herein.

FIGS. 3A-3D provide representative compressive strain-stress curves of plasticized starch foams (using Method 3); foams were compressively deformed under a 1-cycle process at dry (A, ambient conditions) and wet (B, after soaking in distilled water for 15 mins) states; and foams were subjected to a 3-cycle, loading-unloading compression deformation at dry (C) and wet (D) states.

### DETAILED DESCRIPTION OF THE DISCLOSURE

The present disclosure relates to plasticized composite materials that are insoluble in liquid environments but can absorb a large amount of liquid. The composite materials of the present disclosure can advantageously be insoluble in liquid environments while also imparting desirable mechanical strength and liquid barrier properties. Other advantages of the composites of the present disclosure include a very simple production process. Composites of the present disclosure include, for example, insoluble composites such as a plasticized foam containing at least one polymer having a charge, e.g., an anionic polysaccharide such as a starch, and at least one polymer of opposite charge, e.g., cationic polysaccharide such as chitosan, and a plasticizer such as glycerol and/or propylene glycol.

In some embodiments, the composites can be prepared by combining an expandable polymer having a charge, such as anionic polysaccharide, an oppositely charged polymer, such as a cationic polysaccharide, and water, and expanding the mixture using an expansion treatment, such as microwave expansion or extrusion. Starch, for example, especially amylopectin, can exhibit extensive expansion capacity under thermal processing, including microwave heating and thermal extrusion. The starch can be readily soluble in aqueous solutions.

In some embodiments, the composites can be prepared by combining an expandable polymer having a charge, such as anionic polysaccharide, an oppositely charged polymer, such as a cationic polysaccharide, and water to form a mixture. The mixture can be expanded using an expansion treatment (e.g., microwave expansion or extrusion) and a plasticizer immersion process. The preparation can be a two-step process in which the mixture is subjected to the expansion treatment to produce a starch dry foam. Furthermore, after the expansion treatment, the dry foam can be subjected to plasticization (e.g., water immersion), freezing, and lyophilizing. During plasticization, the dry foam can be immersed into a plasticizer solution at an elevated temperature (e.g., of about or above 30° C.) for a predetermined immersion time period (e.g., ranging from about 1 hour to about 2 days). This immersion time can advantageously precondition the material properties of the foam, and are therefore important conditions that can affect mechanical characteristics (e.g., compressive modulus) of the foam. In some embodiments, the predetermined immersion time period can range from 1 hour to about 2 days (e.g., from about 1 hour to about 36 hours, from about 1 hour to about 24 hours, from about 1 hour to about 12 hours, from about 1 hour to about 10 hours, from about 1 hour to about 8 hours, from about 1 hour to about 6 hours, from about hour to about 4 hours, from about 1 hour to about 2 hours, from about 12 hour to about 48 hours, from about 24 hour to about 48 hours, from about 2 hour to about 12 hours, from about 6 hour to about 12 hours, from about 4 hour to about 10 hours, from about 2 hour to about 6 hours, from about 18 hour to about 24 hours).

After the immersion, produced wet foams can be subjected to freezing (e.g., at about −80° C.) and freeze-drying (e.g., at about −50° C., about 0.03 mbar) for a suitable duration (e.g., about or at least 2 days).

The composites can be prepared by combining at least one polymer having a charge (e.g., an anionic polymer), at least one oppositely charged polymer (e.g., cationic polymer), at least one plasticizer, and at least one solvent to form a mixture (e.g., solution), exposing the mixture to an elevated temperature, and freezing and lyophilizing the mixture. In some embodiments, the composites are not subjected to any immersion

The composites can be prepared by combining at least one polymer having a charge (e.g., an anionic polymer), at least one oppositely charged polymer (e.g., cationic polymer), and at least one solvent to form a mixture (e.g., a first solution), exposing the mixture to an elevated temperature, soaking the mixture in a solution (e.g., a second solution), and freezing and lyophilizing the mixture.

The composites can be prepared by combining at least one polymer having a charge (e.g., an anionic polymer), at least one oppositely charged polymer (e.g., cationic polymer), and at least one solvent to form a mixture (e.g., a first solution), exposing the mixture to an elevated temperature, soaking the mixture in a solution (e.g., a second solution) containing a plasticizer, and freezing and lyophilizing the mixture.

The composites can be prepared by combining at least one polymer having a charge (e.g., an anionic polymer), at least one oppositely charged polymer (e.g., cationic polymer), and at least one solvent to form a mixture (e.g., a first solution), exposing the mixture to an elevated temperature, soaking the mixture in a solution (e.g., a second solution) containing a cationic or anionic polysaccharide, and freezing and lyophilizing the mixture. The solution containing the cationic or anionic polysaccharide can advantageously aid in facilitating hemostasis. In some embodiments, the cationic polymer is chitosan. The cationic polymer (e.g., cationic polysaccharide such as chitosan) can be immersed in a plasticizer solution (e.g., glycerol solution) a single soak step. In some embodiments, the cationic polysaccharide can be immersed in a plasticizer solution (e.g., glycerol solution) a multiple soak steps (e.g., in at least two soak steps, or three soak steps) and subsequent freeze-drying steps. In some embodiments, the free concentration of the chitosan in the solution ranges from between 0.5% and 5% of the solution.

The composites can be prepared by combining at least one polymer having a charge (e.g., an anionic polymer), at least one oppositely charged polymer (e.g., cationic polymer), and at least one solvent to form a mixture (e.g., solution), exposing the mixture to an elevated temperature, soaking the mixture in a solution containing a cationic or anionic polysaccharide and plasticizer, and freezing and lyophilizing the mixture.

The anionic polymers and/or cationic polymers can be polysaccharides. Polysaccharides can be obtained in the form of a liquid, gel, powder, matrix, or sphere-like particle. For example, cellulose can be used as described herein in the form of microcrystalline cellulose, microfibrillated cellulose, or hydrolyzed cellulose nanofibers or nanowhiskers, or sphere-like cellulose produced by bacteria including Acetobacter xylinum. Cellulose in sphere-like form can range in size from about 50 μm to about 25000 μm (e.g., 200 μm to 1000 μm, 500 μm to 5000 μm, or 1000 μm to 10000 μm).

Polysaccharides can be obtained from a naturally-occurring starting material or can be produced synthetically. For example, the polysaccharides of a composite provide herein can be obtained from plants (e.g., grasses and trees), animals (e.g., tunicates), or microbes (e.g., Acetobacter xylinum bacteria). In some cases, the polysaccharides of a composite provide herein can be obtained commercially. For example, various grades of cellulose can be obtained from paper and pulp manufacturers such as International Paper, Georgia Pacific, or Weyerhaeuser, or distributors such as Fluka, Sigma Aldrich, and other companies.

In some cases, a polysaccharide provided herein can exhibit various degrees of alignment. For example, cellulose fibrils can be aligned using a magnetic field, an electric field (e.g., a DC or AC electric field), an electromagnetic or optical field, or using fluid flow, where the long axis (along the α-1,4 glucan chain) of the fibrils are generally parallel. Such a configuration can be achieved by applying an electric field to a solution of cellulose fibers or to an active growing culture of microbes (such as the bacteria Acetobacter xylinum) producing cellulose. Such an arrangement can improve the physical properties of the cellulose or any cellulose containing materials and can be used in tissue regeneration applications where growing cells need to grow primarily in one dimension (e.g., along the fiber length). An example of such tissue is nerve tissue (e.g., spinal cord tissue after a break where the break is larger than about 10 μm to about 100 μm).

The composites provided herein can include one or more anionic polymers, for example, an anionic polysaccharide. The polysaccharide can be starch. The anionic polysaccharides can be an anionic starch (e.g., potato starch), amylopectin, amylose, carboxymethyl cellulose, alginic acid, pectin, xanthan gum, hyaluronic acid, carrageenan, xylan, chondroitin sulfate, gum arabic, gum karaya, gum tragacanth, or combinations thereof. In some embodiments, the anionic starch can include amylopectin, amylose, and combinations thereof. The anionic polysaccharide can be a chemically modified starch. The polysaccharide can be cellulose, such as a microbial cellulose. The anionic polysaccharide can be a chemically modified cellulose. The polysaccharide can be an anionic starch such as an anionic amylopectin or a starch that contains phosphate. The starch can contain one phosphate ester group per approximately 20 to 400 anhydroglucose units). The polysaccharide can be chitin.

The composites provided herein can include one or more cationic polymers. The cationic polymer can be a cationic polysaccharide. Suitable cationic polysaccharides can include, but are not limited to chitosan, cationic guar gum, cationic hydroxyethylcellulose and chemically modified starches. The cationic polysaccharide can be a chemically modified cellulose. The cationic polysaccharide can have a molecular weight ranging of at least 10 kDa (e.g., from about 10 kDa to about 2,000 kDa, from about 50 kDa to about 1,000 kDa, from about 100 kDa to about 500 kDa, from about 200 kDa to about 300 kDa, of at least 50 kDa, of at least 100 kDa, of at least 200 kDa, of at least 300 kDa, of at least 500 kDa, or of at least 1,000 kDa).

In some embodiments, the anionic polysaccharide (e.g., starch) comprises at least 70% w/w amylopectin (e.g., at least 75% w/w amylopectin, from about 70% to about 95% w/w, or from about 75% to about 80% w/w). In some embodiments, the anionic starch contains amylopectin present in an amount of at least 75% and the cationic polysaccharide is between 2% and 10%. In some embodiments, the anionic starch comprises at least 20% w/w amylose.

In some embodiments, the ratio of said one or more anionic polysaccharides to said one or more cationic polysaccharides is between about 10:1 to about 50:1. In some embodiments, the ratio of said one or more anionic polysaccharides to said one or more cationic polysaccharides is at least 20:1. In some embodiments, the ratio of said one or more cationic polysaccharides to the plasticizer is about 1:10 to about 1:40.

The composite provided herein can be formed at a pH ranging from about 2 to about 13 (e.g., from about 2 to about 10, from about 2 to about 8, from about 2 to about 7, from about 2 to about 6, from about 2 to about 4, from about 4 to about 13, from about 4 to about 11, from about 4 to about 9, from about 4 to about 7, from about 4 to about 6, from about 5 to about 7, from about 6 to about 7, from about 7 to about 13, from about 8 to about 13, from about 9 to about 13, from about 11 to about 13, from about 4 to about 11, or from about 6 to about 8). In some embodiments, the composition can be formed at a pH that is greater than 7.0. The composite can be formed at a pH between 9.0 and 11.0. In some embodiments, the pH is between 7.0 and 11.0 or a pH between 9.5 to 11.0) is achieved using NaOH and water, or alkaline water. In some embodiments, the cationic polymers and the anionic polymers are combined in a polar solution with a pH between the lowest pKa of the anionic end group and the highest pKa of the cationic end group of the cationic polymers and the anionic polymer. The polar solution can comprise water and formic acid adjusted to a pH of about 3-4. In some embodiments, the solutions can be adjusted to a pH of about 2 to about 6.5, or about 2.5 to 5.5, using acidic water.

The composites provided herein include a plasticizer. Suitable plasticizers include, but are not limited to, glycerol (Gly), propylene glycol (PG), and combinations thereof. In some embodiments, it is preferable to use a plasticizer in an amount of about 25 wt. % to about 95 wt. %. In some embodiments, it is preferable to use a plasticizer having a boiling point between about 150 and about 250° C. In some embodiments, the plasticizer has a structure similar to glycerol except that it may also contain a hydrophobic end group. In some embodiments, the plasticizer is biocompatible. In some cases, the composites provided herein include a plasticizer and a starch that has not been gelatinized to improve the characteristics of the expanded composite including the degree of expansion during thermal treatment which would result in a lower density insoluble composite.

The solvent can be an aqueous solvent, such as water. In some embodiments, at least one pH modifier, such as formic acid or sodium hydroxide, can also be added to the solvent (e.g., to produce acidic water or basic water) to obtain a desired pH level. In some embodiments, the solvent can be a polar solvent. The solvent can be present in an amount between 35% and 85% in the mixture used to form the composite.

In some cases, the use of a soak and freeze-drying technique is provided.

The composites can be prepared by immersing a formed foam (e.g., dry foam) containing at least one anionic polysaccharide , at least one cationic polysaccharide, at least one plasticizer, and at least one solvent to form a mixture at an acidic pH (e.g., a pH of about 2), a neutral pH (e.g., pH of about 7) or a basic pH (e.g., a pH of about 12) environment for a desired time frame to form a wet foam. In some cases, the foam is immersed for at least 1 hour (e.g., at least 2 hours, at least 6 hours, at least 12 hours, at least 24 hours, at least 2 days, at least 5 days, or at least one week). The foam is immersed at a temperature from about 25° C. to about 35° C. (e.g., about 30° C.).

The composites can be prepare by heating a solution containing at least one anionic polysaccharide, at least one cationic polysaccharide, at least one plasticizer, and at least one solvent to form a mixture to a temperature of about 60° C. to about 100° C. (e.g., from about 70° C. to about 90° C., from about 60° C. to about 90° C., from about 80° C. to about 100° C.).

The composites can be prepared by freezing the foam (e.g., a wet foam) formed by a foam formed from a solution containing at least one anionic polysaccharide, at least one cationic polysaccharide, at least one plasticizer, and at least one solvent. The foam can be subjected to freezing by exposing the foam to a temperature of less than 0° C. (e.g., about −80° C.) until a constant temperature is reached.

The composites can be prepared by lyophilizing the foam formed by a solution containing at least one anionic polysaccharide, at least one cationic polysaccharide, at least one plasticizer, and at least one solvent. Lyophilizing can be performed using any conventional, known methods, for example, lyophilizing methods as described in U.S. Pat. No. 7,521,187.

The composites can be prepare by immersing a solution containing at least one anionic polysaccharide, at least one cationic polysaccharide, at least one plasticizer, and at least one solvent to form a mixture in an acidic pH (e.g., a pH of about 2), a neutral pH (e.g., pH of about 7) or a basic pH (e.g., a pH of about 12) environment for a desired time frame. In some cases, the solution is immersed for at least 1 hour (e.g., at least 2 hours, at least 6 hours, at least 12 hours, at least 24 hours, at least 2 days, at least 5 days, or at least one week).

In some embodiments, the composite provided herein is nontoxic to humans.

Other additives can also be included in the composite. In some embodiments, certain component additives may be more suitable for specific applications. For example, collagen may provide a benefit for biomedical applications. In addition, various additives such as antimicrobial and therapeutic agents can be added before or after the heating, the freezing, and/or the lyophilizing step. Therapeutic agents include compounds such as polyhexamethylene biguanide (PHMB) or any contained in U.S. Patent Application 20110150972, 2011. If added as a solution after the heating step by soaking the composite in solution or spraying a solution onto the composite, the composite can be subsequently dehydrated by freeze drying to permit long term storage. The use of chitosan in the composite may provide some measure of natural antimicrobial properties.

### EXAMPLES

### Example 1—Starch Composite Foams

Exemplary composite foams were prepared using the following components:

i) Potato starch (PS) with approximately 75% amylopectin and 25% amylose obtained from Western Polymer;

ii) High purity chitosan (CS) (ChitoCleaer, Primex ehf, Iceland) was obtained with an average molecular weight of 214 kDa, a degree of deacetylation of 90%, and a viscosity of 75 cP (1 wt % solution at 25° C.);

iii) High purity (>99.5%) plasticizers (PS) including glycerol (Gly) and propylene glycol (PG) were obtained from Sigma Aldrich; and

iv) Formic acid (88 wt. %) was obtained from Alfa Aesar, for the purposes of adjusting the pH.

Exemplary composite foams were prepared as described below.

### 1. Preparation of a Standard (Control) Starch Foam

Powders of 4 g potato starch (“PS”) and 0.16 chitosan (“CS”) were homogeneously mixed in a Teflon cup. Afterwards, 3.2 g of acidic water was added, mixed with the dry powders, and the mixture was kneaded into a dough. The weight ratio of these components was shown in Table 1 (PS control), and the total weight of a dough was approximately 7.35 g. The as-prepared dough was then thermally expanded for 48 s in a conventional microwave (LG Electronics Inc., 2450 MHz) at 100% output powder. The thermally expanded dry foam was cut into a cubic shape by removing the outer hard shell and used as a control sample.

### 2. One-Step Preparation of Plasticized Starch Foams (Method 1)

The preparation of a plasticized starch foam was only conducted in the microwave and thus it was defined as one-step prepared starch foam. Similar to the preparation of a standard starch foam, the potato starch, chitosan, and acidic water were combined in a Teflon cup. Additionally, the plasticizer solution was added, mixed, and the mixture was kneaded into a dough. The weight ratio of these components is shown in Table 1 (PS-Gly and PS-PG). Due to the decreased amount of water in the plasticized dough, the microwave expansion time for a dough was correspondingly reduced to 22 s to obtain a soft foam sample. Finally, the foam was cut to remove the outer shell and stored in a plastic bag before compressive test.

### 3. Two-Step Preparation of Plasticized Starch Foams (Method 2)

The two-step preparation of a plasticized starch foam was performed by combining microwave expansion and plasticizer immersion processes. The initial dry starch foam was prepared following the procedures of a standard starch foam. Subsequently, the as-obtained starch dry foam was immersed into a plasticizer solution at 30° C. for 2 days. The concentrations of different plasticizer solutions are shown in Table 1. After a two-day immersion, the wet foams were subject to freezing in a refrigerator (−80° C.) overnight, and then freeze-drying using a Labocon freeze dryer (−50° C., 0.03 mbar) for 2 days. The dried, plasticized foams were stored in a plastic bag before being subjected to a compressive test described herein.

### 4. Two-Step Preparation of Plasticized Starch Foams (Method 3)

4 g potato starch, 45.12 g acidic water, 6 g glycerol and 0.16 g chitosan solution were mixed in a beaker and mechanically homogenized using an Ultra Turrax (IKA. T25) at 20k rpm for 2 min. The mixture was thermally expanded in a conventional microwave for 90 s and was removed from the microwave every 30 s and mixed for 10 s manually using a spatula. This was repeated until a correct solid content (8%, w/v) was achieved. The as-prepared mixture consisted of partially gelatinized starch and the composition is shown in Table 1. Afterwards, the mixture was degassed, poured into a Teflon petri dish, freezed in a refrigerator (−80° C.) overnight and finally lyophilized using a Labocon freeze dryer (−50° C., 0.03 mbar) for 3 days. The as-prepared plasticized starch foam was coded as PS-CS-Gly. For comparison purposes, the plasticized starch foam without chitosan crosslinking (PS-Gly) was prepared following the same procedure.

### 5. Compressive Properties of Starch Foams

Compressive properties of starch foams were evaluated at ambient conditions using a DMA Q800 dynamic mechanical analyzer (TA, Instrument). Square shaped foam samples were prepared and compressed at a constant strain rate of 10% /min and a preload force of 0.01 N. A cyclic loading-unloading experiment was also conducted using the same instrument to study the resiliency properties of foams. Tests were performed at a strain rate of 10%/min to 50% of the compressive strain, followed by an unloading process to a small load of 0.01 N. This loading-unloading cycle was repeated for three times. Three replicates for each starch foam sample were measured and the average value is reported.

TABLE 1
Formulation and preparation process of starch foams

| | | Foam Formulation | | | | |
|---|---|---|---|---|---|---|
| | | Weight ratios | | | | |
| | | | | Acidic | | |
| | | | | water | | Foam preparation |
| Sample | | PS | CS | (pH~2.3) | Plasticizer | method |
| Plasticized | PS control | 25 | 1 | 20 | — | Microwave at 48 s |
| foams | PS-Gly | 25 | 1 | 14 | 14 | Micro wave at 22 s |
| (Method I) | PS-PG | 25 | 1 | 12.5 | 12.5 | Microwave at 22 s |
| Plasticized | PS water | 25 | 1 | 20 | — | Microwave at 48 s, soak |
| foams | immersed | | | | | in water at 30° C. for 2 d, |
| (Method 2) | | | | | | freeze, and lyophilize |
| | PS-Gly | 25 | 1 | 20 | 25 | Microwave at 48 s, soak |
| | immersed | | | | | in 7.5% Gly solution at |
| | | | | | | 30° C. for 2d, |
| | | | | | | freeze, and lyoohilize |
| | PS-PG | 25 | 1 | 20 | 6.25 | Microwave at 48 s, soak |
| | immersed | | | | | in 15% PG solution at |
| | | | | | | 30° C. for 2 d. |
| | | | | | | freeze, and lyophilize |
| Plasticized | PS-Gly | 25 | 0 | 282 | 37.5 | |
| foams | PS-CS-Gly | 25 | 1 | 282 | 37.5 | Microwave at 90 s, |
| (Method 3) | | | | | | degass, mold casting, |
| | | | | | | freeze. and lyophilize |

PS: potato starch, CS: chitosan, Gly: glycerol.

### 6. Solubility of Starch Foams

The pH of DI water was adjusted to 3 with hydrochloride acid and to 9 with sodium hydroxide. Various formulated foams were soaked into the above solutions and DI water (pH˜7) for 30 days to test their solubility. Different levels of solubility, i.e., completely intact, small particles observed, large particles observed, partial disintegration, full disintegration and completely solubilized, were used to qualitatively access the water solubility performance of starch foams.

### Results

### 1. Mechanical Properties

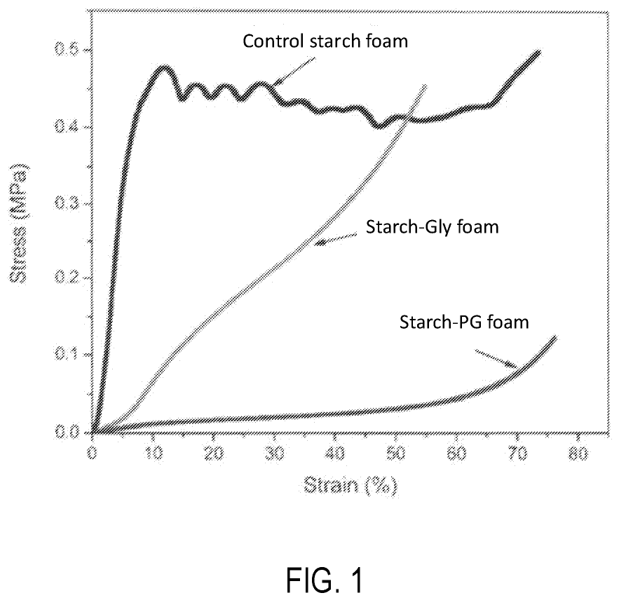

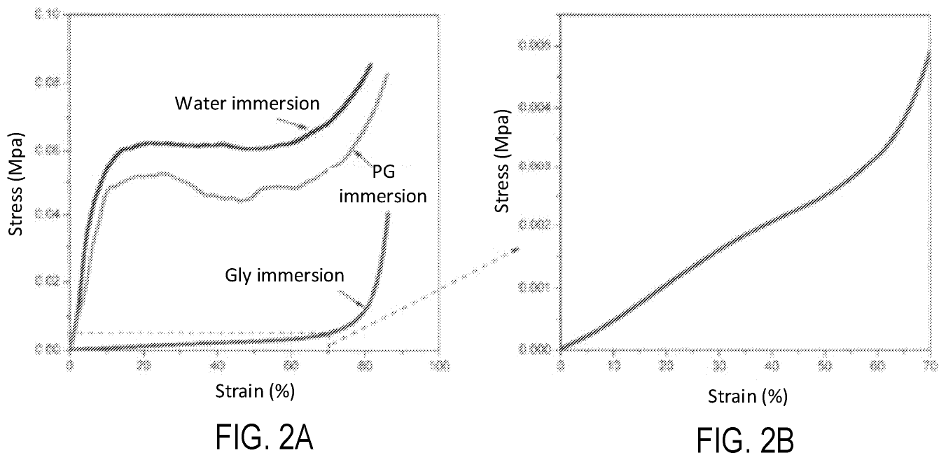

The compressive strain-stress curves of one-step (Method 1) and two-step (Method 2) prepared starch foams are presented in FIG. 1 and FIG. 2, respectively, and the compressive moduli were summarized in Table 2.

TABLE 2
Compressive modulus of plasticized starch foams (Methods 1 & 2)

| | Compressive modulus |
|---|---|
| Sample | (MPa) |

| | | |
|---|---|---|
| Plasticized foams | PS control | 7.73 ± 1.54 |
| (Method 1) | PS-Gly | 0.63 ± 0.33 |
| | PS-PG | 0.11 ± 0.06 |
| Plasticized foams | PS water immersion | 0.89 ± 0.21 |
| (Method 2) | PS-Glv immersion | 0.018 ± 0.0026 |
| | PS-PG immersion | 0.48 ± 0.1 |

Generally, for a typical foam material, three different stages occur during the compression process. Initially, the foam is elastically compressed, showing a linear-elastic region and indicating the bending of the cell walls. Afterwards, a plateau-like stage appears, suggesting the collapse of cell walls. Finally, a steeply increased stress occurs when the foam is further compressed to a higher strain, corresponding to densification process.

Herein, the standard starch foam without any plasticization treatment showed a much higher compressive modulus than the plasticized foams, as presented in Table 2. With the addition of plasticizer, the modulus of starch foam could be modulated, depending on the way of plasticizer incorporation, plasticizer type, and plasticizer content.

For one-step prepared starch foams, PS-Gly and PS-PG displayed compressive moduli of 0.63±0.33 and 0.11±0.06 MPa, respectively, which were much lower than that of an un-plasticized starch foam (7.73±1.54 MPa). The incorporation of these plasticizers could decrease the rigidity of cell walls of a foam. Besides, during the foaming process, water acts as a blowing agent and its evaporation provides the porous structure of the resulting foam. The less water content and interaction between water and plasticizer induced the size shrinkage of the plasticized foam when compared to a standard foam. The differences in porosity, cell size, bulk density, and plasticizer characteristics could explain the variation in compression modulus of tested foams.

Alternatively, the two-step preparation (method 2) of starch foam was chosen and compared with the one-step approach. The thermally expanded and plasticization processed foams possessed very low compressive moduli, suggesting the additional plasticization effect from the lyophilization process. The compressive modulus of a standard starch foam after water immersion was 0.89±0.21 MPa, which was almost an order of magnitude lower than the foam without water immersion (7.73±1.54 MPa). It is possible that large pore and thin cell wall could be formed during the sublimation of ice crystals. Starch foam immerse in PG solution showed a similar stress-strain curve and slightly decreased compressive modulus (0.48±0.1 MPa), ascribed to the low residual PG content (˜25%) in the foam. Attempts were made to immerse starch foams into PG solutions with different concentration s (7.5%, 15%. 20%) and immersion time (2 d, 3 d, 4 d), yet the final PG content in a freeze-dried foams were less than 30%. On the other hand, starch foam immersed in a Gly solution showed a very low compressive modulus (0.018±0.0026 MPa). The freeze drying process could not sublimate the glycerol and thus more glycerol (˜100%) could be maintained in the final foam and make it softer. Also, the freeze drying process may not disturb the location of the glycerol molecules within the starch and chitosan molecules as would thermal drying, making the foams softer and more stable.

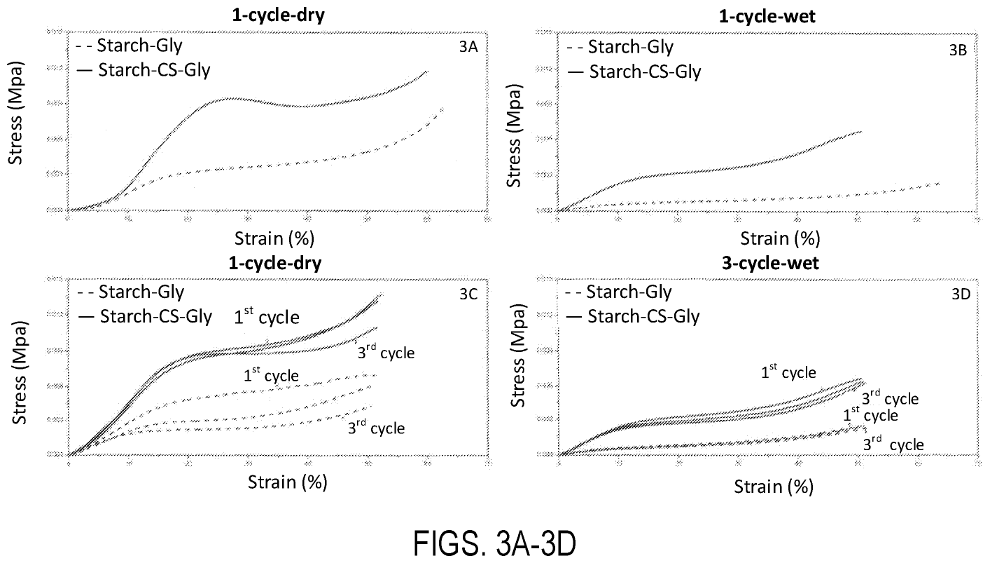

Compressive strain-stress curves of two-step prepared starch foams by Method 3 are shown in FIG. 3, and the compressive moduli were summarized in Table 3. Three typical regions include initial linear-elastic, plateau-like, and densification regimes were observed for all foam samples. At dry state, the compressive moduli of PS-Gly and PS-CS-Gly were 23.6±4.1 and 48.4±6.2 kPa, respectively. The impact of chitosan crosslinking was obvious, reflected by an increase of 105% in compressive modulus. The improvement of compressive modulus of cross-linked foam could be because of strong ionic interaction between anionic starch and cationic chitosan. When foams were subjected to soaking in water for 15 min, water penetrated into the cell walls, and swelled and plasticized the cell walls, resulting in a dramatical decrease in compressive modulus. At wet state, the compressive modulus of PS-Gly and PS-CS-Gly were 6.1±2 and 25.8±3.5 kPa, which were reduced by 74.2% and 47% as compared to those in dry state. It is hypothesized that water could swell PS-Gly to a significantly higher degree due to the hydrophilic nature of gelatinized starch. Due to the ionic crosslinking, the decrease in compressive modulus for PS-CS-Gly was less. The water solubility data shown in Table 4 also indicated that crosslinked starch foam was more resistant to water swelling and plasticization.

To evaluate the resiliency properties, three compression loading-unloading cycles up to 50% compressive strain were performed at dry and wet states as shown in FIGS. 3 (c) and (d), respectively. At dry state, the compression moduli of PS-CS-Gly were quite consistent, indicating a good resiliency under cyclic compressive deformation. The compression modulus of PS-CS-Gly, determined at the third compression cycle, was 46.5 35 7.0 kPa. On the other hand, for uncross-linked PS-Gly foam, the first loading cycle exhibited the highest compressive modulus. The compressive modulus decreased slightly (16.1 ±1.8 kPa) for the subsequent cycles with the same strain. This decrease could be due to the collapse or the breakage of the pore structure of foam. At wet states, both PS-Gly and PS-CS-Gly foams exhibited consistent compressive strain-stress curves as shown in FIG. 3(d).

It should be noted that compression properties of the foam can be influenced by a variety of factors, such as density, pore size and structure, reinforcement of the cell struts and cell walls, and crosslinking degree between cell wall materials. The density of the foam prepared can be controlled by adjusting starch concentration during thermal expansion in microwave. A higher starch concentration (>8%) can result in a denser structure with more small pores and thicker cell walls in the foam, contributing to the improvement in compressive modulus and yield strength. With a lower starch concentration (<8%), the foam would have lower bulk density with more big pores and thin cell walls in the structure, resulting in a decrease in compression properties.

The crosslinking degree is another important aspect. Anionic starch feedstock with a higher degree of substitution (DS) (DS is 0.04 in this example) would induce more ionic interaction sites between starch and chitosan, contributing to the improvement in both compression properties and water solubility. The DS is the number of substituted anionic or cationic groups per sugar residue in the polysaccharide. For example, a DS of 0.04 means that 1 glucose molecule in 25 glucose molecules has a phosphate group in an anionic potato starch. A carboxylate starch with DS 0.1 was used to prepare the foams following the same protocol as described in section 2.4. However, both uncross-linked and cross-linked foams were very sensitive to water and could immediately be soluble in water. This behavior was explained by the low molecular weight of starch feedstock. Therefore, a sufficiently high molecular weight starch with high DS is ideal.

TABLE 3
Compressive modulus of plasticized starch foams (method 3)

| | 1-cycle compressive modulus | | 3-cycle compressive modulus | |
|---|---|---|---|---|
| Sample | Dry (KPa) | Wet (KPa)^{a} | Dry(KPa)^{b} | Wet(KPa)^{a, b} |
| PS-Gly | 23.6 ± 4.1 | 6.1 ± 2.0 | 16.1 ± 1.8 | 5.9 ± 1.6 |
| PS-CS-Gly | 48.4 ± 6.2 | 25.8 ± 3.5 | 46.5 ± 7.0 | 23.4 ± 2.4 |

^{a}: moduli were measured by DMA after soaking foam sample in distilled water for 15 min.
^{b}: moduli were determined at the third cycle

### 3.2 Water Solubility

Table 4 summarizes the water solubility of various starch foams in water solution with different pH values ranging from 3 to 9.

TABLE 4
Water solubility of starch foams

| Sample | | pH = 3 | pH = 7 | pH = 9 |
|---|---|---|---|---|
| PS without CS | | full | full | full |
| | | disintegration | disintegration | disintegration |
| Plasticized | PS control | small particles | small particles | small particles |
| foams | | observed | observed | observed |
| (Method 1) | | | | |
| | PS-Gly | insoluble | insoluble | insoluble |
| | | (completely | (completely | (completely |
| | | intact) | intact) | intact) |
| | PS-PG | insoluble | insoluble | insoluble |
| | | (completely | (completely | (completely |
| | | intact) | intact) | intact) |
| Plasticized | PS water | small particles | small particles | small particles |
| foams | immersion | observed | observed | observed |
| (Method 2) | | | | |
| | PS-Gly | insoluble | insoluble | insoluble |
| | Immersion | (completely | (completely | (completely |
| | | intact) | intact) | intact) |
| | PS-PG | small particles | small particles | partial |
| | immersion | observed | observed | disintegration |
| Plasticized | PS-Gly | full | full | full |
| foams | | disintegration | disintegration | disintegration |
| (Method 3) | | | | |
| | PS-CS-Gly | insoluble | insoluble | insoluble |
| | | (completely | (completely | (completely |
| | | intact) | intact) | intact) |

As a reference, the starch foam without chitosan crosslinking was also prepared. After a 30-day immersion, the starch foam without chitosan crosslinking was completely disintegrated and collapsed due to the hygroscopic feature of gelatinized starch. All the crosslinked foams could maintain their shapes after 30-day water immersion, but varied in the solubility levels depending on foam processing and plasticizer type in the foams.

For one-step prepared foams, only PS control showed small disintegrated particles, and the plasticized foams arc completely insoluble after 30-day water immersion. It is hard to observe the leaching out of plasticizer from the foams for plasticized samples (PS-Gly and PS-PG) after immersing in water for such a long time. Besides, the stiffness of one-step prepared foams after 30-day soaking in water solution are in the following order: PS-Gly>PS-PG>PS control. Both density and porosity could play a role in determining the stiffness of a foam. In the presence of plasticizer, the foam was expected to expand less due to the reduced mobility of water molecule, resulting in a denser and less porous foam. The density and porosity of one-step processed foams will be further examined to prove our hypothesis.

For two-step prepared foams using Method 2, only PS-Gly foam was insoluble and completely intact, while both PS-water and PS-PG foams showed different levels of disintegration after 30-day water immersion. Additionally, all two-step prepared foams (Method 2) could rehydrate and expand into gel-like materials almost immediately after squeezing out the water. For two-step prepared foams using Method 3, crosslinked PS-CS-Gly was completely insoluble under any pH conditions during the test, while uncross-linked PS-Gly was fully disintegrated at every pH condition.

However, foams made using Method 3 were fragile and would break apart more easily if mechanically disturbed. For example, foams made using Method 2 can be compressed driving out a liquid and return to their prior shape while foams made from Method 3 are destroyed in such a process.

### Other Embodiments

It is to be understood that, while the invention has been described herein in conjunction with a number of different aspects, the foregoing description of the various aspects is intended to illustrate and not limit the scope of the invention, which is defined by the scope of the appended claims. Other aspects, advantages, and modifications are within the scope of the following claims.

Disclosed are methods and compositions that can be used for, can be used in conjunction with, can be used in preparation for, or are products of the disclosed methods and compositions. These and other materials are disclosed herein, and it is understood that combinations, subsets, interactions, groups, etc. of these methods and compositions are disclosed. That is, while specific reference to each various individual and collective combinations and permutations of these compositions and methods may not be explicitly disclosed, each is specifically contemplated and described herein. For example, if a particular composition of matter or a particular method is disclosed and discussed and a number of compositions or methods are discussed, each and every combination and permutation of the compositions and the methods are specifically contemplated unless specifically indicated to the contrary. Likewise, any subset or combination of these is also specifically contemplated and disclosed.

What is claimed is:

1. An insoluble composite material formed by:
creating a mixture of at least one anionic polysaccharide, at least one cationic polysaccharide, at least one plasticizer, and at least one solvent;
heating the mixture;
pouring the mixture into a mold;
freezing the mixture; and
lyophilizing the mixture.

2. An insoluble composite material formed by:
creating a mixture containing at least one anionic polymer, at least one cationic polymer, and at least one solvent;
exposing the mixture to an elevated temperature to reduce the content of the solvent to solidify the mixture;
soaking the solid mixture in a second solution containing a plasticizer;
freezing the mixture; and
lyophilizing the mixture.

3. An insoluble composite material formed by:
creating a mixture containing at least one anionic polysaccharide, at least one cationic polysaccharide, and at least one solvent;
exposing the mixture to an elevated temperature to reduce the content of the solvent to solidify the mixture;
soaking the solid mixture in a second solution containing a plasticizer;
freezing the mixture; and
lyophilizing the mixture.

4. An insoluble composite material formed by:
creating a mixture containing at least one non-gelatinized anionic starch, at least one chitosan, and at least one aqueous solvent;
exposing the mixture to an elevated temperature to gelantinize the starch and reduce the content of the solvent to solidify the mixture;
soaking the solid mixture in a second solution containing a plasticizer;
freezing the mixture; and
lyophilizing the mixture.

5. An insoluble composite material formed by:
creating a mixture containing at least one non-gelatinized anionic starch, at least one chitosan, and at least one aqueous solvent;
heating the mixture to partially gelantinize the at least one non-gelatinized anionic starch;
pouring the mixture into a mold;
freezing the mixture; and
lyophilizing the mixture.

6. An insoluble composite material formed by:
creating a mixture containing at least one anionic polysaccharide, at least one cationic polysaccharide, and at least one solvent;
heating the mixture to reduce solvent content in the mixture and to form a solid mixture;
soaking the solid mixture in a second solution containing a plasticizer and a charged polysaccharide;
freezing the mixture; and
lyophilizing the mixture.

7. An insoluble composite material formed by:
creating a mixture containing at least one anionic polysaccharide, at least one cationic polysaccharide, and at least one solvent;
heating the mixture to reduce solvent content in the mixture and to form a solid mixture;
soaking the solid mixture in a second solution containing a plasticizer and an antiseptic agent (e.g., antiseptic molecule such as PHMB);
freezing the mixture; and
lyophilizing the mixture.

8. An insoluble composite or coating material comprising a mixture of at least one anionic polysaccharide, at least one cationic polysaccharide, and a solvent and a plasticizer.

9. The insoluble composite material of any one of claims 1-6, wherein said plasticizer is a molecule that contains carbon, oxygen and hydrogen; a molar mass between 60 and 95: a boiling point between 150° C. and 300° C.; at least one —OH group; at least one CH2 group; and/or is nontoxic to humans.

10. The insoluble composite material of any one of claims 1-9, wherein said plasticizer is glycerol, propylene glycol or combinations thereof.

11. A method for producing a composite or coating composition, wherein the method comprises:
combining one or more anionic polysaccharides, one or more cationic polysaccharides, a plasticizer, and a solvent to obtain a solution,
heating the solution,
freezing the mixture; and
lyophilizing the mixture.

12. The method of claim 11, wherein said one or more anionic polysaccharides comprises at least one anionic starch.

13. The method of claim 12, wherein said at least one anionic starch comprises amylopectin.

14. The method of claim 12 or 13, wherein the anionic starch is selected from the group consisting of amylopectin, amylose, and combinations thereof.

15. The method of any one of claims 12-14, wherein the anionic starch comprises at least 70% w/w amylopectin and at least 20% w/w amylose.

16. The method of any one of claims 11-15, wherein said one or more cationic polysaccharides is chitosan.

17. The method of any one of claims 11-16, wherein the ratio of said one or more anionic polysaccharides to said one or more cationic polysaccharides is between about 10:1 to about 50:1.

18. The method of claim 17, wherein the ratio of said one or more anionic polysaccharides to said one or more cationic polysaccharides is at least 20:1.

19. The method of any one of claims 11-18, wherein said plasticizer comprises glycerol, propylene glycol or combinations thereof.
